(19) United States
(12) Patent Application Publication
(10) Pub. No.: US 2022/0233853 A1
(43) Pub. Date: Jul. 28, 2022

(54) SYSTEMS AND METHODS FOR TREATING TUMORS USING TARGETED NEUROSTIMULATION

(71) Applicants: Emiliano SANTARNECCHI, Boston, MA (US); Alvaro PASCUAL-LEONE, Wayland, MA (US); Neuroelectrics Corporation, Cambridge, MA (US)

(72) Inventors: Giulio RUFFINI, Brookline, MA (US); Emiliano SANTARNECCHI, Boston, MA (US); Alvaro PASCUAL-LEONE, Wayland, MA (US)

(21) Appl. No.: 17/610,246

(22) PCT Filed: May 20, 2020
(86) PCT No.: PCT/US20/33842

§ 371 Date: Nov. 10, 2021

Related U.S. Application Data
(60) Provisional application No. 62/850,310, filed on May 20, 2019.

(51) Int. Cl. A61N 1/36; A61B 5/00; A61B 5/055; A61N 1/04; A61N 1/20; A61N 1/05

(52) U.S. Cl. CPC A61N 1/36002; A61B 5/0042; A61B 5/055; A61B 5/4836; A61B 2562/04; A61N 1/205; A61N 1/0408; A61B 5/6814; A61N 1/0531; A61N 1/0476

(57) ABSTRACT

A method for treating diagnosed or suspected tumors of a subject, comprising obtaining a target map that identifies an actual or likely location of the tumor in the subject, and providing multifocal non-invasive electrical stimulation with a duration, spatiotemporal pattern, current intensity, electrode montage, and/or regimen sufficient to do one or more of the following: (1) reduce the size of one or more tumor(s), (2) alter its/their perfusion, (3) change its/their metabolic or electrical activity, (4) change its/their functional connectivity profile, (5) slow down or stop its/their progression/spread and related symptomatology, (6) characterize one or more tumor(s) based on its/their response to noninvasive brain stimulation; possibly in conjunction with optimized anatomical changes applied to the skin or skull to better steer currents and fields into the tumor.

### PRIORITY

This application claims priority to and benefit from U.S. Provisional Patent Application Ser. No. 62/850,310, filed on May 20, 2019, the entire contents of which are incorporated by reference herein.

### FIELD OF THE INVENTION

The present invention relates to methods for treating brain tumor and other tumors. The methods use maps and models of the brain (or other parts of the body) to provide non-invasive electrical stimulation targeted to brain or body locations having, suspected of having, or at risk for having tumors, and may include opportunistic or intentional electro-anatomical features in the tissues relevant to current conduction in the tumor—such as skin preparation or small skull holes —to better channel electrical currents into the tumor. This targeted electrical stimulation reduces the vascular perfusion of the tumor, slowing, stopping or reversing the growth of the tumor, or in some embodiments preventing or reducing the spread of metastases. In some aspects, the method identifies and locates tumors by evaluating tissue response to electrical stimulation. The various aspects and embodiments of the invention may be used both before and/or after surgery (e.g., brain surgery).

### BACKGROUND

Glioblastoma and metastasis represent the most aggressive and frequent adult brain neoplasm, with a median survival of only 12 months despite the aggressive treatment, including neurosurgery, chemotherapy, and radiotherapy. Glioblastoma (GBM) accounts for more than 60% of adult brain tumors (Rock et al., 2012). It is considered as grade IV of the glioma lineage in the WHO classification (Louis et al., 2016), affecting approximately 10 per 100,000 people in the world (Hanif, Muzaffar, Perveen, Malhi, & Simjee, 2017). Together with brain metastasis, GBM represents the majority of brain neoplasm affecting the human adult brain and, despite aggressive treatment combining neurosurgery, radiotherapy and pharmacological approaches, patients still show a median survival window after the diagnosis of about only 12 months (Ganbold et al., 2017).

Electro-perfusion therapy (EPT) is a therapeutic modality for bodily tumor treatment based on the application of a low intensity direct electric current directly into the tumor by means of two or more platinum electrodes inserted into the tumor, or indirectly by electrodes placed in the surrounding tissue (Gina et al., 2013). EPT can be performed in association with chemotherapy (electrochemotherapy-ECT), leveraging its effect on tumor trans-membrane permeability and consequent increasing the number of drugs penetrating into the tumor cells (Hills & Stebbing, 2014). The first clinical application of EPT was done on lung cancer patients by Nordenstrom (Nordenstrom, Eksborg, & Beving, 1990) and after preliminary positive results, ET has been tested on a few malign superficial and visceral tumors, such as hepatocellular carcinoma (Fosh et al., 2003), advanced breast cancer and breast hemangioma (Yoon et al., 2007) and pancreas carcinoma (Wu, Zhou, & Huang, 2001) (for a review see (Ciria et al., 2013). Results in vivo confirmed findings previously obtained with in vitro experiments, showing a reduction of the tumor mass or even the absence of tumor recurrence, with minimal side effects (Ciria et al., 2013).

Despite these encouraging results, EPT has not been used on brain tumors, possibly because of the invasive surgical procedure through the skull required to insert the stimulation electrodes into the tumor mass, similar to traditional surgical resection.

### SUMMARY OF THE INVENTION

The present invention is based, in part, on the discovery of methods for treating and/or preventing tumors in the brain. The methods use target maps to provide non-invasive brain stimulation specifically to brain locations having, suspected of having, or at risk for having tumors.

Aspects and embodiments of the invention employ transcranial current stimulation (tCS, also sometimes referred to as transcranial electric stimulation, tES). tCS is a form of non-invasive brain stimulation (NIBS) that uses electrodes placed on the scalp to deliver electrical currents to the brain (Ruffini 2013). tCS is used to stimulate or inhibit one or more target brain region(s). tCS includes a family of related non-invasive techniques such as direct (tDCS), alternating (tACS), random noise current stimulation (tRNS), or any other form of multichannel current stimulation. This includes a variant where each electrode may be configured to stimulate with a unique, independent, and arbitrary waveform only limited by current conservation, creating spatiotemporal patterns in the quasi-static approximation regime, with frequency spectra band-limited to <10 kHz). tCS can be used to stimulate one circumscribed brain region as well as to engage multiple regions composing a “brain network” (Ruffini, Wendling, Sanchez-Todo, & Santarnecchi, 2018). This approach could be used to stimulate the network of brain regions connected to the tumor, to reduce tumor propagation and/or modulate intratumoral activity/perfusion. Electro-perfusion therapy differs from other forms of electrotherapy in that its mechanism of action is the reduction of perfusion. It specifically differs from the application of high frequency electric fields to interfere with mechanisms of cellular reproduction, as with Tumor Treating Fields (TTFields).

In some aspects, the present invention provides methods for reducing one or more tumor(s) in the brain of a subject. The methods include steps of obtaining a target map, which identifies actual location(s) of the tumor(s) and/or likely location(s) for tumor (s) and providing a non-invasive brain stimulation with a duration, frequency spectrum, current intensity, electrode montage, and/or regimen sufficient to (1) reduce the size of one or more tumor(s), (2) alter its/their perfusion, (3) change its/their metabolic or electrical activity, (4) change its/their functional connectivity profile, (5) slow down or stop its/their progression/spread and related symptomatology, and/or (6) characterize one or more tumor(s) based on its/their response to noninvasive brain stimulation. Methods may also include the alteration of electro-anatomical characteristics of surrounding tissues to influence current flow.

In other aspects, the present invention provides methods for slowing, stopping or reversing growth of one or more tumor(s) in the brain of a subject. The methods include steps of creating a target map comprising actual location(s) of the tumor(s) in the subject's brain and densities thereof and/or likely location(s) of tumor(s) in the subject's brain, determining appropriate transcranial current stimulation (tCS) stimulation parameters to target the actual location(s) and/or likely location(s) of the tumor(s), and providing a non-invasive brain stimulation under the appropriate tCS stimulation parameters to target the actual location(s) and/or likely location(s) of the tumor(s). Consequently, the methods (1) reduce the size of one or more tumor(s), (2) alter its/their perfusion, (3) change its/their metabolic or electrical activity, (4) change its/their functional connectivity profile, (5) slow down or stop its/their progression/spread and related symptomatology, (6) characterize one or more tumor(s) based on its/their response to noninvasive brain stimulation.

In some embodiments, the methods include steps of creating a target map comprising actual location(s) of tumor(s) in a subject's brain and/or likely location(s) of tumor(s) and determining appropriate non-invasive brain stimulation parameters to target the actual location(s) and/or likely location(s) of the tumor(s). The targeting of the actual location(s) of the tumor with the appropriate non-invasive brain stimulation (NIBS) stimulation parameters would reduce the size of the tumor(s).

Non-invasive brain stimulation NIBS includes tCS and its specific variants (tDCS, tACS, tRNS and others), transcranial magnetic stimulation (TMS) and its specific implementations (including single pulse, monophasic or biphasic, repetitive or burst TMS, etc.), and FUS (focused ultrasound), or any other form of stimulation that can be applied non-invasively to interact with neuronal populations. As used herein, the term “transcranial current stimulation” or “tCS” (also sometimes referred to as “transcranial electric stimulation” or “tES”) includes all its variants, including the variant where each electrode may be configured to stimulate with its own unique, independent, and arbitrary waveform, only limited by current conservation.

In embodiments, an optimization procedure is used to target tCS generated fields on the cortex of a subject's brain. Computational models of brain function and dysfunction may play a key role in reducing risk and uncertainty in clinical trials and provide the means for personalized therapies that account for individual biophysical and physiological characteristics. This can be achieved by incorporating a mechanistic understanding of the effects of tCS within realistic brain models, thereby enabling the effective development of synergistic, individualized therapies. Aspects of the procedures have been described in WO2015/059545 and U.S. Pat. No. 9,694,178, which are hereby incorporated by reference in their entireties. Also described therein are uses of optimized multichannel transcranial current stimulation that preferentially engage the target map; this target map includes the locations that propagate activation signals upon stimulation, locations that propagate inhibition signals upon stimulation, and neutral locations, which may be avoided.

In embodiments, optimal currents, optimal electrode locations, and/or optimal electrode numbers are determined using a realistic head model with electric field modeling. In embodiments, the electric field calculations are performed using the realistic head model described in Miranda et al., (2013). In embodiments, the realistic head model is a multilayer finite element model of a realistic head that may be either generic or specific to a patient, e.g., from an MRI of the patient or other brain imaging technology.

In embodiments, the tCS delivers a frequency spectrum in the quasi-static regime (<˜10,000 Hz) to the location. In embodiments, the frequency spectrum delivered is 0 Hz (i.e., tDCS), in others it is in the gamma band, e.g., 25 Hz and about 100 Hz. In embodiments, the frequency spectrum is between about 40 Hz and about 50 Hz. See U.S. 62/630,685, which is hereby incorporated by reference in its entirety.

In embodiments, the stimulation includes more than one distinct frequency spectra, e.g., including at least one frequency spectrum in the gamma band or at least one frequency spectrum in the gamma band and at least one frequency spectrum outside the gamma band. In embodiments, each frequency spectrum is in the gamma band. In embodiments, at least one frequency spectrum is a non-sinusoidal waveform, e.g., the non-sinusoidal waveform is in the gamma band. In embodiments, the stimulation includes random and/or varying frequencies.

In embodiments, the stimulation has a current intensity between about 0.1 mA and about 10 mA or between about 0.01 A/m² to about 100 A/m².

In embodiments, the stimulation has a duration of at least 1 second, at least 1 minute, or at least 1 hour. In some embodiments, the stimulation duration is at least 2 hours. In various embodiments, the stimulation duration is from about 5 minutes to about 2 hours, or from about 5 minutes to about 1 hour.

In embodiments, the regimen (of tCS) includes only one session. Alternately, the regimen includes more than one session, with the sessions being annual, bimonthly, monthly, semimonthly, biweekly, weekly, semiweekly, daily, or more than daily, and any number of periodic sessions therebetween.

As used herein, “about” is defined as ±10% of the associated numerical value.

In embodiments, tCS is provided via at least one electrode, and in some embodiments via an electrode montage comprising more than one electrode. In embodiments, at least two electrodes in the electrode montage have different stimulation parameter (including frequency spectra and/or intensities). In embodiments, each electrode in the electrode montage has the same stimulation parameters. The electrode montage may include up to 2, up to 4, up to 8, up to 16, up to 32, up to 64, up to 128, or up to 256 electrodes. The electrode montage may include from about 1 to about 300, about 1 to about 250, about 1 to about 200, about 1 to about 150, about 1 to about 100, about 1 to about 50, about 1 to about 40, about 1 to about 30, about 1 to about 25, about 1 to about 20, from about 1 to about 15, from about 1 to about 10, from about 2 to about 8, or from about 4 to about 8 electrodes. In some embodiments, the electrode montage comprises at least 2, at least 4, at least 8, or at least 16 electrodes. The electrode may include about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, or about 35 electrodes. In some embodiments, the electrode montage comprises from 2 to 32 electrodes, or from 4 to 16 electrodes, or from 4 to 8 electrodes.

In embodiments, the subject has detectable tumor(s) in his/her brain and has clinical or preclinical disease. Alternately, the subject does not have detectable tumors in his/her brain and the methods provide protection for a subject at risk, including a subject at risk for recurrence.

In embodiments, the subject does have detectable tumors in his/her brain and the methods provide information on the location of the tumor(s), information on the type of tumor (e.g. glioblastoma, metastasis) and information on its aggressiveness.

In embodiments, multichannel stimulation is optimized together with a specification of minimal anatomical changes, such as skin preparation specific to some electrodes, or as drilling small diameter skull holes, creating thinner skull areas or placing conductive material around/inside the tumor mass to control electrical field.

Any aspect or embodiment described herein can be combined with any other aspect or embodiment as disclosed herein.

Finally, the techniques described focus on tCS, but can also be applied, to some extent with single or multichannel TMS.

### BRIEF DESCRIPTION OF THE DRAWINGS

FIGS. 1A, 1B, 1C, 1D, 1E, and 1F illustrate an example of a workflow in accordance with embodiments of the present invention. FIG. 1A depicts T1w and T2-weighted images that are acquired as part of the standard presurgical diagnostic iter. FIG. 1B depicts Gadolinium Contrast-Enhanced (CE) T1w images that are used to localize the tumor, which is then manually segmented in two tissue classes (solid tumor and necrotic core), while edema is also identified on a FLAIR MRI scan. FIG. 1C depicts MRI images and regions-of-interest that are used to create personalized tCS models and stimulation montages. FIG. 1D depicts a combined tCS-MRI session where ASL and fMRI sequences are acquired before, during and after stimulation that uses the personalized tCS models and stimulation montages of FIG. 1C. FIG. 1E depicts schematically that brain surgery is performed according to standard clinical procedures, and subsequent anatomopathological examination is conducted to label the tumor histologically. FIG. 1F shows that a second MRI, as well as tCS-MRI session, are performed in a post-surgical stage, accounting for skull breaches might alter current diffusion. FLAIR=Fluid Attenuated Inversion Recovery; tCS=transcranial Current Stimulation; ASL=Arterial Spin Labeling; fMRI=Functional Magnetic Resonance Imaging.

FIGS. 2A, 2B, 2C, and 2D. An example of potential modeling, segmentation and Individualization of stimulation montages. FIG. 2A: MRI images are manually segmented, identifying regions of interest (ROIs) representing the edema (green), solid tumor (red, GBM in this example) and necrotic core (blue).

FIG. 2B: Conductivity values are assigned to each ROIs as well as to healthy brain tissue (grey and white matter, CSF, skin, skull). FIG. 2C: a multi-electrode stimulation solution is implemented to maximize stimulation over the edema-solid tumor interface. FIG. 2D: The resulting tCS montage including up to 8 electrodes placed over the hemisphere homolateral to the tumor.

FIGS. 3A, 3B, and 3C. An example of Modeling Pipeline for Post-surgical Stimulation. FIG. 3A: Structural MRI and CT images are used to model the impact of tCS after surgery. Ad-hoc ROIs are created for both skull breaches and metallic clips that could respectively favor current shunting and affect electrodes positioning. FIG. 3B: New tissue conductivity values are derived, and the amount of current shunting through skull breaches is carefully estimated. FIG. 3C: In the case of biopsy, morphology of the tumor (glioblastoma in this example) and amount of edema might be significantly altered after surgery, resulting in a modification of the estimated induced electric field and into a different multifocal electrode montage.

FIGS. 4A and 4B. Impact of non-invasive brain stimulation on tumor perfusion and tumor size. FIG. 4A: Significant reduction in white matter-corrected CBF was observed during stimulation for both patients with GBM and MTX (p.<0.01), as compared to a trending change in the edema (p.<0.12) and no changes in the necrotic core (p.<0.39). FIG. 4B: Control ROIs in the homo-and ipsi-lateral hemispheres to each tumor did not show significant changes in CBF. In the figures, GBM denotes Glioblastoma; MTX denotes metastasis; tCS denotes transcranial Current Stimulation.

FIGS. 5A, 5B, and 5C. Geometry of elements used in modeling of heads with implants or holes for improved calculation of currents and electric fields generated by transcranial stimulation. FIG. 5A shows an image of a skull with titanium skull plates and gold EEG electrode. FIG. 5B shows burrhole cover CAD model (left panel) and a plate on top of a hole (right panel). FIG. 5C shows images of a skull, a dog-bone plate CAD model, an an image of a plate as seen from the side.

FIGS. 6A, 6B, and 6C. Finite element head model of one of the patients participating in the present study. Conductivities of the different tissues in the head model (FIG. 6A), including edema, solid tumor and necrotic core regions in the glioblastoma. Magnitude of the E-field (in V/m) in the GM, WM and glioblastoma tissues induced by the montages obtained with the surface optimization (FIG. 6B) and volume optimization (FIG. 6C) approaches.

FIG. 7. Finite element head model of one of the patients participating in the present study. Magnitude of the E-field (in V/m) in the GM, WM and glioblastoma tissues induced by the montages obtained with the surface optimization approach. The skull with 4 craniotomy openings is shown overlaid on top of the image.

### DETAILED DESCRIPTION OF THE INVENTION

The present disclosure demonstrates that glioblastoma tumor perfusion and tumor size can be reduced by targeted non-invasive brain stimulation. In some embodiments, the invention is performed with patients with Glioblastoma or brain metastasis who are selected for brain surgery; the tCS intervention for modulation of perfusion may take place both before and/or after brain surgery; different methodological aspects are considered for the two scenarios, concerning the modeling of current distribution in presence of skull breaches after surgery.

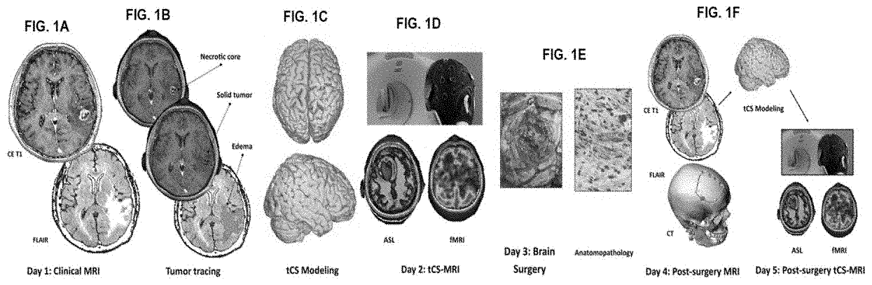

First, (i) for the pre-surgery MRI acquisition (FIGS. 1A-1F) patients underwent a clinical MRI evaluation in order to define and characterize the brain tumor (Langen, Galldiks, Hattingen, & Shah, 2017), including T1-Weighted with and without contrast enhancing agent (T1, CE-T1, respectively), T2-Weighted (T2W), fluid-attenuated inversion recovery (FLAIR), Arterial Spin Labeling (ASL) and resting-state T2-BOLD functional MRI (rs-fMRI). After the visualization of the MRI images, the team decides if the patient is eligible for the study. In case of a positive evaluation, (ii) individual Region of Interest (ROIs) would be segmented by parcellating the solid component of the tumor when present (i.e. active proliferating malignant cells (Urbahska, Sokolowska, Szmidt, & Sysa, 2014, Rees, Smirniotopoulos, Jones, & Wong, 1996), its necrotic core (i.e. the “death” part of the tumor caused by its uncontrolled proliferation that outgrown its blood supply (Rees, Smirniotopoulos, Jones, & Wong, 1996); (Urbahska, Sokolowska, Szmidt, & Sysa, 2014) using CE-T1 sequences, and the edema surrounding the tumor (i.e. infiltrative edema in case of GBM and tumor-free vasogenic edema for metastasis (Z.-X. Lin, 2013) (Stummer, 2007)) by using FLAIR images. The ROIs would be used to create an (iii) individual tCS template, based on the individualized computational models of the transcranial current flow through tumoral and healthy brain tissue space. A dedicated MRI session would be planned over the 3-5 days preceding the surgical intervention (iv), and tCS would be performed inside the MRI scanner by means of an MRI-compatible device Roughly 1 week after the pre-surgery MRI, (v) patients would undergo the neurosurgery/biopsy of the tumor, with subsequent anatomopathological response (vi) to confirm the suspected diagnosis.

The structural MRI (T1-weighted) of each patient was preprocessed using an ad-hoc pipeline in order to obtain individual stimulation solutions. Cortical grey matter surfaces were obtained and then intersected with the edema, solid and necrotic tumor masks, defining a target region aimed at maximizing electric field over the intersection of the edema and solid tumor mask. This target region was then used as input to an optimization algorithm (Stimweaver algorithm, (Ruffini, Fox, Ripolles, Miranda, & Pascual-Leone, 2014)) based on a genetic algorithm comparing every multi-electrode montage composed by up to 8 stimulating electrodes located on any of the 32 positions of the 10-10 EEG system. The objective of the optimization was to maximize the stimulation in the target region by using the rational that an electric field normal to the cortical surface (E_n) and going into it leads to stimulation of the underlying cortical pyramidal cells (Ruffini, Fox, Ripolles, Miranda, & Pascual-Leone, 2014). Solutions were found using constrained least squares comparing weighted target and E_n-field cortical maps to optimize current intensities. In this particular case, the target E_n-field was set to 0.25 V/m in the target area, using PITRODE electrodes (cylindrical 1 cm radius, πcm2 area Ag/AgCl/gel electrode), a maximal current at any electrode of 2.0 mA, a maximal total injected current of 4.0 mA and 8 electrodes or less.

Before starting the MRI-tCS session, patients were comfortably seated in a chair sited in the MRI anamnesis room. Scalp was gently cleaned with alcohol solution to improve skin conductivity under the corresponding electrode. An MRI-compatible brain stimulator was used for the tCS session (Starstim system, with multi-channel MRI kit, Neuroelectrics, Barcelona) and patients wore the device through the entire MRI session.

Approximately one week after the first MRI evaluation patients received brain surgery at the Neurosurgery department of Le Scotte Hospital of Siena (LL, GO). Complete resection (CR, defined as the resection of more than the 98% of tumor (Ahmadloo et al., 2013)) was performed on X patients, subtotal resection (STR, defined as the resection between the 50% and 98% of tumor) on X, partial resection (PR, resection of less the 50% of the tumor) on X and biopsy on the remaining X. Patients did not report any intraoperative complications as well as at the post-operative CT scan controlling for post-operative bleeding, tension pneumoencephalon, ischemia, or brain swelling (Lin, Pay, Naidich, Kricheff, & Wiggli, 1977). Fragments of parenchymal tissue including the lesions removed during the surgery were sent to the anatomopathological department to obtain the diagnosis. Immunohistochemical detection of IDH1, p53, MIB-1 as EGFR expression and MGMT promoter methylation status were performed as part of the routine assessment.

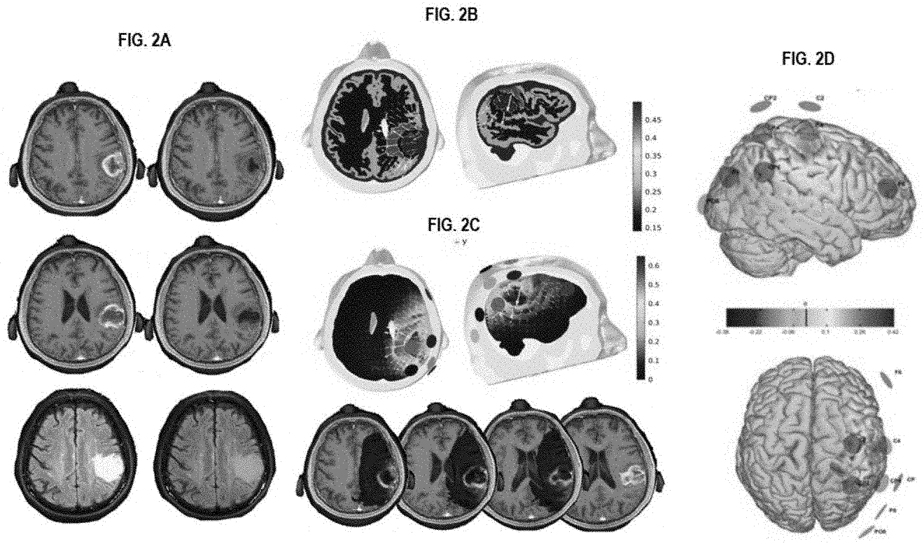

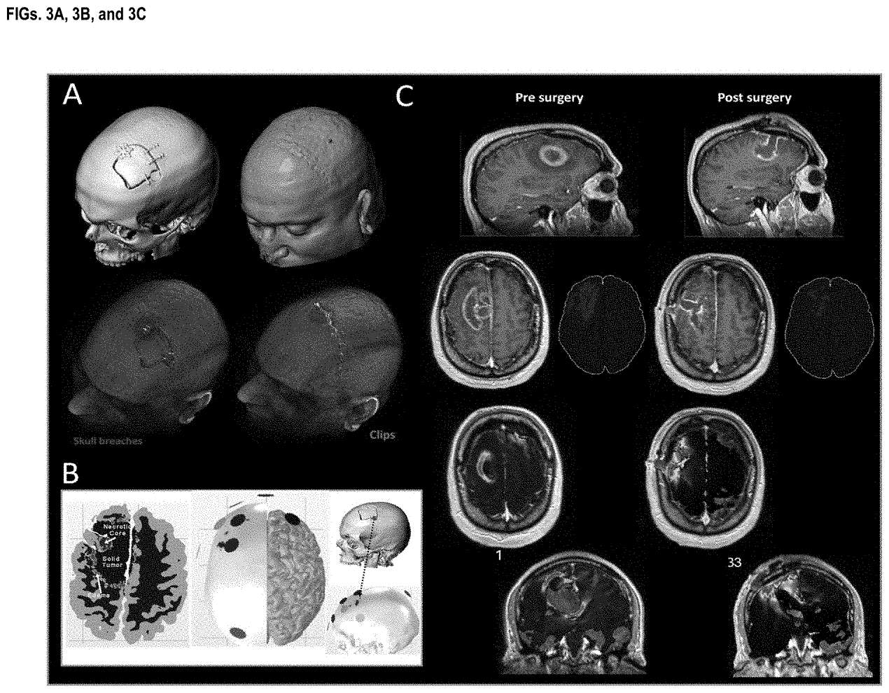

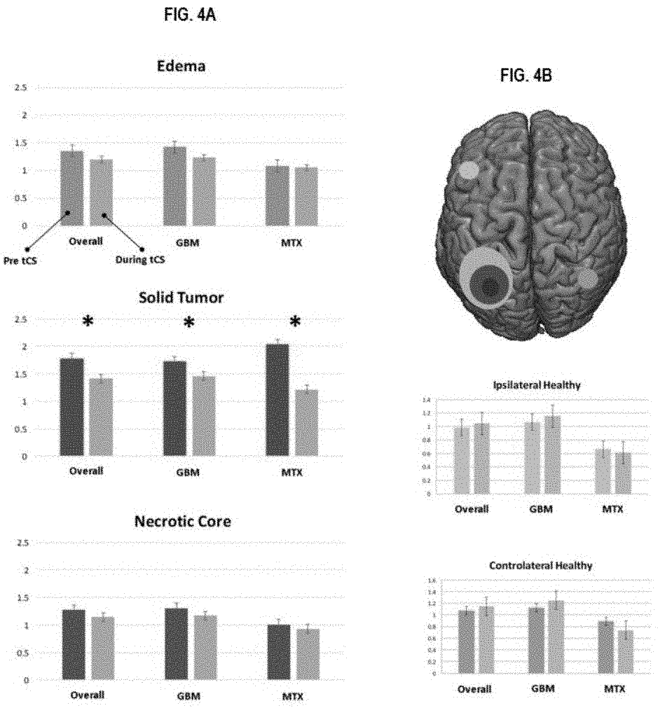

In a first exploratory study on 10 patients with brain tumor, the procedure described above and shown in FIGS. 1A-1F, 2A-2D, and 3A-3C has led to a significant change in tumor's perfusion during tCS, as compared to the changes observed in not-stimulated tumor/healthy tissue. More specifically (FIGS. 4A and 4B), cerebral blood flow (CBF) values for Necrotic core showed a mean decrease of about −19%. In particular, GBM patients displayed a decrease of about −20%, while MTX patients reported −9% decrease. As for the Solid tumor, a mean decrease of −19% was reported, with a −13% and −45% decrease for, respectively, GBM and MTX patients. As for the Edema, a mean −16% decrease was observed, with −17% and −8% for GBM and MTX. The analysis of change in perfusion in healthy control brain regions revealed an opposite pattern, with an overall increase in perfusion of +19% (contralateral ROIs) and +5% (ipsilateral ROIs).

In embodiments, the process of producing an optimized montage for a patient includes three different steps. The first step involves constructing a realistic head model of the patient, which includes accounting for any skull and brain lesions in post-surgery patients. This is important for safety, since skull defects can channel currents and amplify the electric field by creating “hot spots”, but also to preserve the fidelity of the simulation. Finite element models can be created based on individual structural MRI data and manually traced regions of interest representing edema, solid tumor and necrotic core.

The second step is the definition of the target. Broadly, this refers to the weighted selection of target regions or masks, with a specification of the desired electric field characteristics on them. For example, strong electric fields can be desired to be generated on or around the solid tumor, but not on the rest of the cortex. The output of this step is the definition of a cost function for optimization, as discussed in more detail below.

The third step of the process is optimization. Once the head model and the targets are defined, the best electrode and current montage can be found, with “best” mathematically defined by the cost function. To achieve a high spatial resolution, tCS solutions based on multiple stimulation scalp electrodes can be found using a genetic algorithm that seeks to minimize the cost function, as described in Ruffini et al., 2014 (Stimweaver algorithm). The process of optimization may also include using or inducing electro-anatomical changes, such as small holes in the skull due to surgery. In order to do this, such changes needs to be properly modeled, as discussed below.

In some embodiments, the invention involves modeling subjects with craniotomies: Skull lesions (holes) and implants (thin epicranial plates) that can severely impact current flow in tDCS. These should be modeled for safety and efficacy but may also be used to better channel currents and fields into the tumor.

Previous studies have shown that the presence of skull openings can lead to 220% increases of the induced E-field in the brain (although still keeping the E-field within safety limits), also leading to important differences in overall E-field distribution. Accurate modeling of these craniotomies (and any metal implants that may be present) is therefore crucial to accurately and safely plan stimulation approaches.

To refine prior studies and focus on particular intervention elements, embodiments of the present disclosure provide a realistic finite element model of craniotomies for optimization of montages in post-surgery patients. The realistic finite element model is created using safety considerations. With regard to safety, to consider various scenarios (e.g., “worst-case” scenarios), situations with various skull defect sizes (1.0, 2.5 and 5.0 mm radius, with bone flap gaps of about 2 mm), different values of Ti epicranial plate conductivity (very low to very high), and montages (with a placement of the highest current electrode on top of the defects and nearby positions) are considered. The modeling work has focused on a realistic rendition of plates and skull defects as found in craniotomies (Rotenberg et al., 2007). Calculations are performed as described in Miranda et al. (2003) and in Ruffini et al. (2014). The model contains representations of the scalp, skull, cerebrospinal fluid (CSF) (including ventricles), grey matter (GM) and white matter (WM). The conductivities for each of these tissues were based on reference values found on the literature for DC-low frequency range: 0.33 S/m, 0.008 S/m, 1.79 S/m, 0.4 S/m and 0.15 S/m, respectively. In the models with the skull hole, the latter is represented as CSF (conductivity of 1.79 S/m), which is an accurate description for an acute-type skull lesion. The modeling work conducted by the inventors has shown that, in agreement to prior work with similar scenarios, that cortical electric fields generated in a variety of situations (electrode positions, hole radius, worst case conductivity scenarios) remain below 7 V/m —and well below safety limits from animal studies. This is again in agreement with prior studies and tests, which show minimal heating. To put these values into perspective, these results indicate that the peak dissipative power density in cortical tissue never exceeds 0.02 mW/g. As detailed in Bikson et al. (2016), electrical field modeling indicates that typical tDCS protocols (≤2 mA) administered over a range of skull defects may result in local increase in current density compared to the intact-skull case, under worst-case theoretical conditions corresponding to a six-fold increase in brain current density [Datta 2010] (equivalently, electric field). Even allowing for current/field amplification factors due to cranial defects or implants, tDCS induced fields in the present study is significantly below what has been shown to cause injury in animal models (Liebetanz 2009). Modeling studies where skull hole-electrode arrangements were designed to maximize the amplification of cortical electric fields achieved amplification factors (˜10) that remained below safety limits (Seo 2017). In all cases (even in overly pessimistic worst-case scenarios), there is a good safety range (3×-10×) in cortical E field intensity as extrapolated from animal models. Power dissipation and heating are negligible in the grey matter and also in the Ti plate.

Nevertheless, this amplification in electric fields and currents can be advantageous to the treatment of tumors.

In the recently completed study at the Siena Medical School and University Hospital (SIENA STUDY), patients were treated using a multichannel tCS montage. Three of these patients were stimulated for 20 minutes (tDCS, 8 channels, with a maximal intensity per electrode of 2 mA) one month after brain surgery, without adverse effects. Careful modeling work was used to optimize the tCS solution to consider skull breaches and avoid electric field hotspots resulting from skull defects, with no adverse effects reported in the three patients exposed to stimulation after surgery.

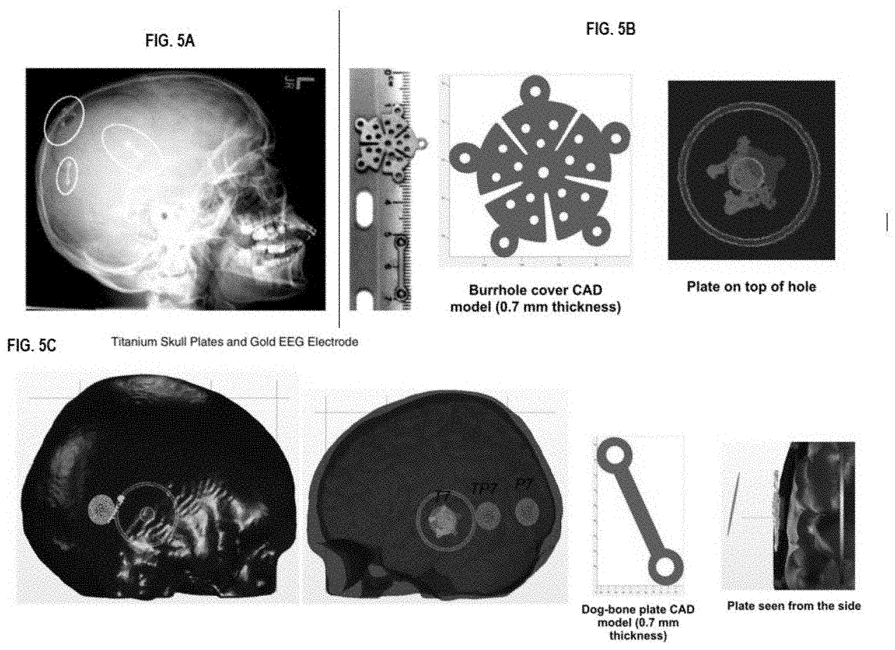

FIGS. 5A, 5B, and 5C illustrate geometry of elements used in modeling of heads with implants or holes for improved calculation of currents and electric fields generated by transcranial stimulation. FIG. 5A shows an image of a skull with titanium skull plates and gold EEG electrode. FIG. 5B shows burrhole cover CAD model (left panel) and a plate on top of a hole (right panel). FIG. 5C shows images of a skull, a dog-bone plate CAD model, an an image of a plate as seen from the side.

At the same time, and as mentioned above, anatomical changes, such as small holes or indentations in the skull, can be artificially created to better channel currents into the tumor. Current flow will basically be favored through a skull hole (for example with an electrode delivering current placed on top of it), or through a region of the skull with a portion (a few mm) carved out, as the skull is a resistive tissue.

Once the model and the masks are defined, one can define cost functions associated with both the edema surface (as described above) or, for example, to the solid tumor volume, to assess the effectivity of each approach. As in earlier work, care is to be taken to avoid electrodes which are on top or near skull lesions to minimize current amplification effects, which will be monitored in the models for safety.

Defining the Optimization Cost Function on Volumes Vs. Surfaces

The optimization goal can be to maximize the electric field normal on edema interface nodes with minimal impact over the rest of the brain, with the physiological goal of increasing perfusion in the edema. The methodology for this approach, which relies on the definition of surface nodes and surface normal vectors, is described at length in Ruffini et al., 2014.

In the present disclosure, it was observed that, as a result of the stimulation, an electric field maximum was generated in the solid tumor volume (which was expected on biophysics grounds, due to the low conductivity of this tissue and its interface with the high conductivity necrotic core), and, crucially, that this led to a decrease of perfusion in the solid tumor. This finding, together with the available animal research data, indicates that a modified targeting strategy, which involves maximizing the electric field magnitude or, equivalently, the power delivered into the solid tumor, can be advantageous. The following notation can be used:

ζ=σ|E|²=J·E (1)

for the electric field power dissipation density (in W/m³), the focus of optimization, with σ denoting the medium conductivity. The basic element for weighted least squares optimization is essentially as in Ruffini et al. 2014, but on volumes rather than surfaces. It is given by the cost function

X(I;ζ⁰,W)=ΣᵢWᵢ²(ζᵢ⁰−ζᵢ(I)²ΔVᵢ, (2)

with the sum over volume nodes, wherein ζᵢ(I) is the electric field energy density as a function of electrode currents, W is a weight function, and ζ⁰ the target electric field energy density at a given node. The last term is the volume element associated with a node. Notably, in the present optimization procedure, the target power dissipation rates are defined for each region, and then the expectations are weighted. This is analogous to the Stimweaver algorithm (Ruffini 2014), but the optimization scheme is shifted to volume rather than surface elements.

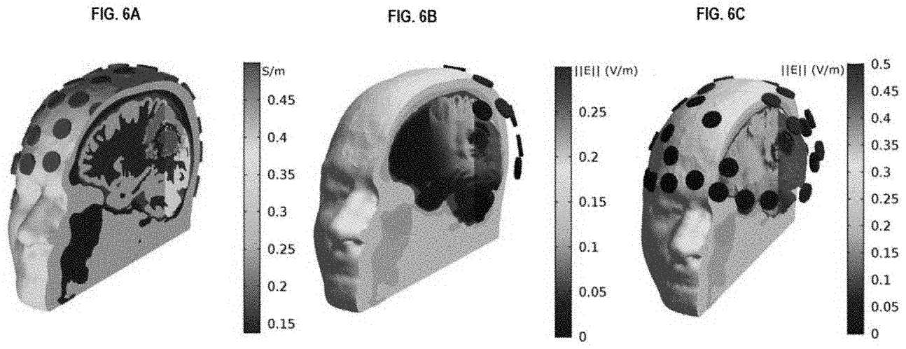

FIGS. 6A-6C display the targets and optimized multielectrode solutions based on the edema surface targeting scheme versus those resulting from the solid tumor volume approach in one of the studied patients. As can be observed, the resulting solutions are different, which is expected given the different associated cost functions. FIGS. 6A-6C illustrate a finite element head model of one of the patients participating in the current study. Conductivities of the different tissues in the head model (FIG. 6A), including edema, solid tumor and necrotic core regions in the glioblastoma. Magnitude of the E-field (in V/m) in the GM, WM and glioblastoma tissues induced by the montages obtained with the surface optimization (FIG. 6B) and volume optimization (FIG. 6C) approaches.

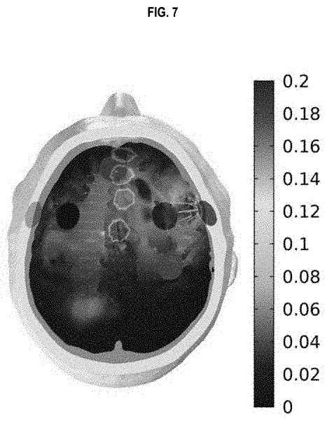

FIG. 7 illustrates a finite element head model of one of the patients participating in the current study. Magnitude of the E-field (in V/m) in the GM, WM and glioblastoma tissues induced by the montages obtained with the surface optimization approach. The skull with 4 craniotomy openings is shown overlaid on top of the image.

Combined Optimization of Electrode Positions, Currents and Anatomical Changes

The current Stimweaver algorithm is capable of searching in the space of electrode positions and currents to identify an optimal configuration matching the desired weighted target map. The method employs a genetic algorithm to carry out this search. This method can be directly applied to head models with electro-anatomical changes, for example, due to prior surgery.

Moreover, this method can simply be extended to expand the search with additional dimensions. Artificial changes in skull thickness or even holes of different diameters can placed at different locations and solutions explored at the same time as currents and electrode positions, for example. The insertion of implants with different conductivity properties can also be explored.

Finally, this method is applicable to other tCS targeting problems, opportunistically or purposefully. It can be applied in epilepsy, for example, where patients may also have undergone surgery.

### EQUIVALENTS

While the invention has been described in connection with some embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties. Exemplary publications are listed below in the References section and throughout the disclosure.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### REFERENCES

Alexiou et al. (2014). Correlation of diffusion tensor, dynamic susceptibility contrast MRI and (99m)Tc-Tetrofosmin brain SPECT with tumour grade and Ki-67 immunohistochemistry in glioma. Clinical Neurology and Neurosurgery, 116,41-45.

Antal et al. (2017). Low intensity transcranial electric stimulation: Safety, ethical, legal regulatory and application guidelines. Clinical Neurophysiology: Official Journal of the International Federation of Clinical Neurophysiology, 128(9), 1774-1809.

Antal et al. (2014). Transcranial electrical stimulation modifies the neuronal response to psychosocial stress exposure. Human Brain Mapping, 35(8), 3750-3759.

Assis et al. Corrosion characterization of titanium alloys by electrochemical techniques. Electrochimica Acta. 2005; 51:1815-9.

Baeken et al. (2017). Increased left prefrontal brain perfusion after MRI compatible tDCS attenuates momentary ruminative self-referential thoughts. Brain Stimulation, 10(6), 1088-1095.

Bikson et al. (2016). Safety of transcranial Direct Current Stimulation: Evidence Based Update 2016. Brain Stimul. Sep.-Oct.;9(5):641-661.

Ciria et al. (2013). Antitumor effects of electrochemical treatment. Chinese Journal of Cancer Research=Chung-Kuo Yen Cheng Yen Chiu, 25(2), 223-234.

Datta et al. (2010). Transcranial direct current stimulation in patients with skull defects and skull plates: High-resolution computational FEM study of factors altering cortical current flow. Neuroimage 52(4): 1268−78.

Fosh et a/.(2003). Use of electrolysis for the treatment of non-resectable hepatocellular carcinoma. ANZ Journal of Surgery, 73(12), 1068-1070.

Ganbold et al. (2017). Differences In High-Intensity Signal Volume Between Arterial Spin Labeling And Contrast-Enhanced T1-Weighted Imaging May Be Useful For Differentiating Glioblastoma From Brain Metastasis. The Journal of Medical Investigation: JMI, 64(1.2), 58-63.

Giordano et al. (2017). Mechanisms and Effects of Transcranial Direct Current Stimulation. Dose-Response: A Publication of International Hormesis Society,15(1), 1559325816685467.

Griffin et al. (1995). Low-level direct electrical current therapy for hepatic metastases. I. Preclinical studies on normal liver. British Journal of Cancer, 72(1), 31-34.

Hanif et al. (2017). Glioblastoma Multiforme: A Review of its Epidemiology and Pathogenesis through Clinical Presentation and Treatment. Asian Pacific Journal of Cancer Prevention: APJCP, 18(1), 3-9.

Hills et al. (2014). Electrotherapy: enlightening modern medicine. The Lancet. Oncology, 15(10), 1060-1061.

Hong et al. (2017). Brain plasticity following MI-BCI training combined with tDCS in a randomized trial in chronic subcortical stroke subjects: a preliminary study. Scientific Reports, 7(1), 9222.

Hu et al. (2012). Correlations between perfusion MR imaging cerebral blood volume, microvessel quantification, and clinical outcome using stereotactic analysis in recurrent high-grade glioma. AJNR. American Journal of Neuroradiology, 33(1), 69-76.

Huang et al. (2016). Response Assessment in Neuro-Oncology Criteria and Clinical Endpoints. Magnetic Resonance Imaging Clinics of North America,24(4), 705-718.

Huang et al. (2017). Measurements and models of electric fields in the in vivo human brain during transcranial electric stimulation. Elife. 2017 Feb. 7;6. pii: e18834. doi: 10.7554/eLife.18834.

Jarm et al. (1999). Blood perfusion of subcutaneous tumours in mice following the application of low-level direct electric current. Advances in Experimental Medicine and Biology, 471,497-506.

Jarm et al. (2003). Perturbation of blood flow as a mechanism of anti-tumour action of direct current electrotherapy. Physiological Measurement, 24(1), 75-90.

Jeck et al. (2018). Bevacizumab in temozolomide refractory high-grade gliomas: single-centre experience and review of the literature. Therapeutic Advances in Neurological Disorders, 11, 1756285617753597.

Jovanovic et al. (2017). Differentiation between progression and pseudoprogresion by arterial spin labeling MRI in patients with glioblastoma multiforme. Journal of B.U.ON.: Official Journal of the Balkan Union of Oncology, 22(4), 1061-1067.

Khadka et al. (2017). Minimal Heating at the Skin Surface During Transcranial Direct Current Stimulation. Neuromodulation. Jan. 22. doi: 10.1111/ner.12554.

Langen et a/.(2017). Advances in neuro-oncology imaging. Nature Reviews. Neurology, 13(5), 279-289. https://doi.org/10.1038/nrneuro1.2017.44.

Liebetanz et al. (2009). Safety limits of cathodal transcranial direct current stimulation in rats. Clin. Neurophysiol. 120,1161-1167.

Li et al. (2006). Effect of electro-acupuncture in treating patients with lingual hemangioma. Chinese Journal of Integrative Medicine, 12(2), 146-149.

Lin et al. (2017). Structural Connectivity Variances Underlie Functional and Behavioral Changes During Pain Relief Induced by Neuromodulation. Scientific Reports,7, 41603.

Lin (2013). Glioma-related edema: new insight into molecular mechanisms and their clinical implications. Chinese Journal of Cancer, 32(1), 49-52.

Lindner et al. (2017). Intraoperative resection control using arterial spin labeling—Proof of concept, reproducibility of data and initial results. Neurolmage. Clinical, 15,136-142.

Louis et al. (2016). The 2016 World Health Organization Classification of Tumors of the Central Nervous System: a summary. Acta Neuropathologica, 131(6), 803-820.

Miranda et al. (2003). The electric field induced in the brain by magnetic stimulation: a 3-D finite-element analysis of the effect of tissue heterogeneity and anisotropy. IEEE Trans. Biomed. Eng. 50, 1074-1085.

Miranda et al. (2013). The electric field in the cortex during transcranial current stimulation. Neuroimage. 2013 Apr. 15; 70:48-58.

Mrugala et al. (2017). Tumor Treating Fields in Neuro-Oncological Practice. Curr Oncol Rep. Aug.;19(8):53.

Nitsche et al. (2000). Excitability changes induced in the human motor cortex by weak transcranial direct current stimulation. The Journal of Physiology, 527 Pt 3,633-639.

Nitsche et al. (2011). Transcranial direct current stimulation—update 2011. Restorative Neurology and Neuroscience, 29(6), 463-492.

Nordenstrom et al. (1990). Electrochemical treatment of cancer. II: Effect of electrophoretic influence on adriamycin. American Journal of Clinical Oncology, 13(1), 75-88.

Opitz et al. (2016). Spatiotemporal structure of intracranial electric fields induced by transcranial electric stimulation in humans and nonhuman primates. Scientific Reports 6:31236.

Petr et al. (2016). Early and late effects of radiochemotherapy on cerebral blood flow in glioblastoma patients measured with non-invasive perfusion MRI. Radiotherapy and Oncology: Journal of the European Society for Therapeutic Radiology and Oncology, 118(1), 24-28.

Plesnicar et al. (1994). Electric treatment of human melanoma skin lesions with low level direct electric current: an assessment of clinical experience following a preliminary study in five patients. The European Journal of Surgery. Supplement.: =Acta Chirurgica. Supplement, (574), 45-49.

Qiao et al. (2015). Arterial spin-labeling perfusion MRI stratifies progression-free survival and correlates with epidermal growth factor receptor status in glioblastoma. AJNR. American Journal of Neuroradiology, 36(4), 672-677.

Rees et al. (1996). Glioblastoma multiforme: radiologic-pathologic correlation. Radiographics: A Review Publication of the Radiological Society of North America, Inc, 16(6), 1413-1438; quiz 1462-1463.

Rock et al (2012). A clinical review of treatment outcomes in glioblastoma multiforme—the validation in a non-trial population of the results of a randomised Phase III clinical trial: has a more radical approach improved survival? The British Journal of Radiology, 85(1017), e729-733.

Rotenberg et al. (2007). Minimal heating of titanium skull plates during 1 Hz repetitive transcranial magnetic stimulation. Clinical Neurophysiology 118.11: 2536-2538.

Rotenberg et al. (2009). Safety of 1 Hz repetitive transcranial magnetic stimulation (rTMS) in patients with titanium skull plates. Clinical Neurophysiology 120.7:1417.

Ruffini et al. (2014). Optimization of multifocal transcranial current stimulation for weighted cortical pattern targeting from realistic modeling of electric fields. Neurolmage. 89:216-25.

Seo et al. (2017). The Effect of a Transcranial Channel as a Skull/Brain Interface in High-Definition Transcranial Direct Current Stimulation—A Computational Study. Scientific Reports 7:40612.

Stagg et al. (2013). Widespread modulation of cerebral perfusion induced during and after transcranial direct current stimulation applied to the left dorsolateral prefrontal cortex. The Journal of Neuroscience: The Official Journal of the Society for Neuroscience, 33(28), 11425-11431.

Stummer (2007). Mechanisms of tumor-related brain edema. Neurosurgical Focus,22(5), E8.

Urbahska et al. (2014). Glioblastoma multiforme —an overview. Contemporary Oncology, 18(5), 307-312.

Wu et al. (2001). [Electrochemical therapy and implanted ports treatment for unresectable carcinoma of body and tail of pancreas]. Zhonghua Wai Ke Za Zhi [Chinese Journal of Surgery], 39(8), 596-598.

Yoon et al. (2007). Introduction of Electrochemical Therapy (EChT) and Application of EChT to The Breast Tumor. Journal of Breast Cancer, 10(2), 162.

Zhang et a/.(2003). Transurethral electrochemical treatment of benign prostatic hyperplasia. Chinese Medical Journal, 116(1), 104-107.

Zheng et al. (2011). Effects of transcranial direct current stimulation (tDCS) on human regional cerebral blood flow. Neurolmage,58(1), 26-33.

Zheng et al. (2016). Modulating transcallosal and intra-hemispheric brain connectivity with tDCS: Implications for interventions in Aphasia. Restorative Neurology and Neuroscience, 34(4), 519-530.

What is claimed is:

1. A method for treating one or more tumor(s) of a subject, comprising:
obtaining a target map, wherein the target map identifies actual location(s) of tumor(s) in a subject and/or likely location(s) of tumor(s) in a subject; and
providing multifocal non-invasive electrical stimulation with a duration, spatiotemporal pattern, current intensity, electrode montage, and/or regimen sufficient to do one or more of the following: (1) reduce the size of one or more tumor(s), (2) alter its/their perfusion, (3) change its/their metabolic or electrical activity, (4) change its/their functional connectivity profile, (5) slow down or stop its/their progression/spread and related symptomatology, (6) characterize one or more tumor(s) based on its/their response to noninvasive brain stimulation; possibly in conjunction with optimized anatomical changes applied to the skin or skull to better steer currents and fields into the tumor.

2. The method of claim 1, where the tumor(s) is located in the brain.

3. The method of claim 2, where the tumor(s) is any tumor recognized by the World Health Organization (WHO), including meningiomas; astrocytoma and oligodendroglioma; including diffuse gliomas; medulloblastomas and other embryonal tumors; including glioblastoma; IDH-wildtype and glioblastoma; IDH-mutant; diffuse midline glioma; H3 K27M-mutant; RELA fusion-positive ependymoma; medulloblastoma; WNT-activated and medulloblastoma; SHH-activated; and embryonal tumor with multilayered rosettes; C19MC-altered; including melanocytic tumors; lymphomas; histiocytic tumors; germ cell tumors; mesenchymal non-meningothelial tumors; tumors of the sellar region; tumors of the pineal regions; choroid plexus tumors; neuronal and mixed-neuronal glial tumors; embryonal tumors; tumors of the cranial and paraspinal nerves; and metastatic tumors.

4. The method of claim 2, wherein the target map defines desired values for the electric field on the cortex surface to maximize stimulation over the edema-solid tumor interface and minimize stimulation over the rest of the brain.

5. The method of claim 4, where the electrode montage has at least 1 electrode and no more than 1024 electrodes.

6. The method of claim 5, wherein the electrode montage has from 2 to 32 electrodes, or optionally from 2 to 16 electrodes, or optionally from 2 to 8 electrodes, or optionally from 4 to 8 electrodes.

7. The method of claim 6, wherein electrodes are arranged according to an EEG 10-20 or 10-10 system.

8. The method of any one of claims 1 to 7, where the form of tCS is selected from one or more of: tDCS, tACS, tRNS or gF-tCS.

9. The method of any one of claims 1 to 8, wherein the target map is based upon a brain image or scan of the subject.

10. The method of claim 9, wherein the image or scan is selected from CT, fMRI, fNIRS, MRI, PET, rs-fcMRl, and SPECT, or a combination thereof.

11. The method of any one of claims 1 to 10, where the target is defined by the intersection of edema and solid tumor mask.

12. The method of any one of claims 1 to 10, where the target is defined by the solid tumor mask.

13. The method of any one of claims 1 to 12, wherein the electric field normal on the interface between the edema and solid tumor masks is maximized with minimal impact over the rest of the brain, or optionally where the quantity to optimize is the magnitude of the electric field, or optionally wherein the electric field magnitude on the solid tumor masks is maximized with minimal impact over the rest of the brain.

14. The method of any one of claims 1 to 13, where tCS is optimized relying on the MRI of the patient.

15. The method of claim 9, wherein the image or scan is selected from EEG, ERPs, MEG, theta-burst rTMS, TMS/EEG, and TMS/MEPs, or a combination thereof.

16. The method of claim 15, wherein EEG and/or MEG is used to determine the stimulation waveform(s) and/or spatiotemporal stimulation pattern.

17. The method of any one of claims 1 to 16, wherein the target map defines a desired spatiotemporal stimulation pattern for the subject.

18. The method of claim 17, wherein an electrode montage is selected to deliver a spatiotemporal stimulation pattern, optionally using a genetic algorithm.

19. The method of claim 18, wherein the target map defines desired values for the electric field on the cortex surface to maximize stimulation over the edema-solid tumor interface and minimize stimulation over the rest of the brain.

20. The method of claim 18 or 19, wherein the genetic algorithm is performed with cross-over and mutation functions, where a binary DNA string specifies at least a montage of electrode number and locations.

21. The method of claim 20, wherein cross-over and mutation functions are defined such that the offspring do not violate a constraint of maximal number of electrodes.

22. The method of claim 20, comprising, performing calculation of optimal currents and electrode number and locations.

23. The method of claim 22, wherein said calculations are performed under constraints regarding at least maximal electrode number and maximal current at each electrode and the total current injected into the brain by all electrodes at any time.

24. The method of any one of claims 1 to 23, wherein the montage includes up to 8 electrodes placed over the hemisphere homolateral to the tumor.

25. The method of any one of claims 1 to 24, wherein the stimulation waveform is in the quasi-static regime of less than about 10,000 Hz.

26. The method of any one of claims 1 to 24, wherein the stimulation waveform is in the DC band.

27. The method of any one of claims 1 to 24, wherein the stimulation waveform is in the gamma band.

28. The method of any one of claims 1 to 27, wherein the stimulation comprises more than one distinct stimulation waveform.

29. The method of any one of claims 1 to 28, wherein the current intensity is from about 0.1 mA to about 10 mA or from about 0.1 A/m² to about 100 A/m².

30. The method of claim 29, wherein the duration is at least 1 second, at least 1 minute, or at least 1 or 2 hours, and optionally is from about 5 minutes to about 1 hour.

31. The method of any one of claims 1 to 30, wherein the regimen comprises at least one session.

32. The method of claim 31, wherein the regimen comprises more than one session, with the sessions being annual, bimonthly, monthly, semimonthly, biweekly, weekly, semiweekly, daily, or more than daily, and any number of periodic sessions there between.

33. The method of claim 32, wherein the tCS is selected from transcranial direct current stimulation (tDCS), transcranial alternating current stimulation (tACS), random noise current stimulation (tRNS), general field stimulation (gF-tCS), or a variant where each electrode is configured to stimulate with σ unique, independent, and arbitrary waveform.

34. The method of claim 33, wherein the tCS is performed before brain surgery.

35. The method of claim 33, wherein the tCS is performed after brain surgery, with generic or individualized modeling of current accounting for skull breaches and potential current modification of induced electrical field in the tumor and surrounding healthy brain tissue

36. The method of claim 33 or 34, wherein the tCS is performed in patients with a tumor and in combination with MRI sequences sensitive to cerebral blood flow, perfusion, blood oxygenation level, neurotransmitters level, white and grey matter structural properties, to localize the tumor mass by looking at tissue response to electrical stimulation.

37. The method of claim 33 or 34, wherein the tCS is performed in patients with a brain tumor and in combination with MRI sequences sensitive to cerebral blood flow, perfusion, blood oxygenation level, neurotransmitters level, white and grey matter structural properties, to estimate tumor's aggressiveness and its potential to spread over surrounding and distant healthy brain tissue, by looking at tissue response to electrical stimulation.

38. The method of claim 33, wherein the tCS is performed in combination with drug therapy (e.g. Chemotherapy) before brain surgery.

39. The method of claim 35, wherein the tCS is performed in combination with drug therapy (e.g. Chemotherapy) after brain surgery.

40. The method of any one of claims 1 to 39, wherein the optimization of multichannel tCS is carried out in conjunction with a specification and model for anatomical changes such as drilling small skull holes or indentations.

41. The method of claim 1, wherein the tumor is a bodily tumor not in the brain.
